(19) United States
(12) Patent Application Publication
(10) Pub. No.: US 2013/0095038 A1
(43) Pub. Date: Apr. 18, 2013

(54) PARTICLES, AND PHOTOACOUSTIC IMAGING CONTRAST AGENT AND SLN CONTRAST AGENT INCLUDING THE PARTICLES

(71) Applicant: CANON KABUSHIKI KAISHA, Tokyo (JP)

(72) Inventors: Mayuko Kishi, Machida-shi (JP); Fumiko Tomatsu, Yokohama-shi (JP); Tatsuki Fukui, Yokohama-shi (JP); Tetsuya Yano, Tsukuba-shi (JP); Kouichi Kato, Yokohama-shi (JP); Mika Yamagata, Tokyo (JP); Satoshi Yuasa, Yokohama-shi (JP)

(73) Assignee: CANON KABUSHIKI KAISHA, Tokyo (JP)

(21) Appl. No.: 13/651,727

(22) Filed: Oct. 15, 2012

(30) Foreign Application Priority Data
Oct. 15, 2011 (JP) 2011-227507
Oct. 15, 2011 (JP) 2011-227508
Oct. 15, 2011 (JP) 2011-227509
Mar. 21, 2012 (JP) 2012-063899

(51) Int. Cl. A61K 49/00

(52) U.S. Cl. 424/9.1

(57) ABSTRACT

Provided are particles where a hydrophilic coloring agent having an anionic functional group, such as ICG, hardly leaks from the particles. The particles include a coloring agent having an anionic functional group, such as ICG, ions of a polyvalent metal, such as iron ions (Fe²⁺ or Fe³⁺), zinc ions, and a polyol compound.

### BACKGROUND OF THE INVENTION

1. Field of the Invention

The present invention relates to particles, and photoacoustic imaging contrast agent and SLN contrast agent including the particles.

2. Description of the Related Art

In recent years, photoacoustic imaging has been attracting attention as a noninvasive diagnostic imaging method.

The photoacoustic imaging is a method of forming an image of an object to be measured by irradiating the object with light and detecting the intensity and the location of acoustic waves generated by volume expansion due to heat emitted by the object's molecules. In the photoacoustic imaging, a coloring agent can be used as a contrast agent for increasing the intensity of acoustic waves from the object site to be measured.

For example, indocyanine green (hereinafter, may be abbreviated as ICG) is known as a coloring agent that emits acoustic waves through absorption of light. Throughout the specification, ICG means compounds having a structure represented by Chemical Formula (1):

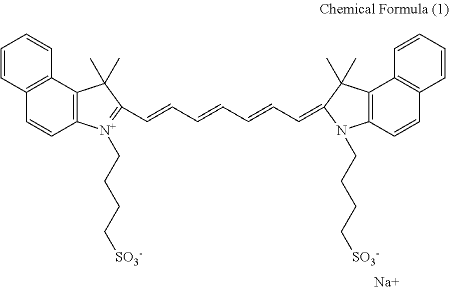

wherein, the counter ion is not limited to Na⁺ and may be H⁺ or K⁺.

Incidentally, Journal of Photochemistry and Photobiology B: Biology, 74 (2004), 29-38 (Non-Patent Literature 1) discloses particles of poly(lactide-co-glycolide) (hereinafter, may be abbreviated as PLGA) containing ICG prepared by an emulsion solvent diffusion method using polyvinyl alcohol (PVA) as a surfactant.

At the same time, in recent years, the detection rate of early-stage cancers has been increased to increase the opportunity of surgery to remove early-stage cancers. In general, in early-stage cancer surgery, it is important to judge whether lymph nodes located near the lesion site (cancer cells) and suspected of metastasis are removed or not during the surgery of the lesion site.

In metastasis of cancer to lymph nodes, random metastasis hardly occurs, and it is suggested that cancer spreads according to a certain pattern from a lesion site, through a lymph vessel, to a lymph node. The first lymph node that receives cancer cells from a primary cancer lesion through a lymph vessel is the sentinel lymph node (throughout the specification, may be abbreviated as SLN). When metastasis to a lymph node is found, it is believed that there is metastasis to SLN on the upper stream of the path in which cancer cells travel.

Accordingly, there is a demand for technology for accumulating a contrast agent at SLN and detecting the SLN with high accuracy.

Incidentally, Japanese Patent Laid-Open No. 2001-299676 (Patent Literature 1) describes an example of using ICG as a fluorescence imaging contrast agent for detecting SLN.

### SUMMARY OF THE INVENTION

Unfortunately, the ICG-containing PLGA particles disclosed in Non-Patent Literature 1 have a problem that ICG leaks in an aqueous solution such as serum, since ICG is a hydrophilic coloring agent having an anionic functional group. Accordingly, aspects of the present invention provide particles of which a hydrophilic coloring agent having an anionic functional group, such as ICG, hardly leak.

In the detection of SLN described in Patent Literature 1, only ICG is administered. The present inventors have found, however, a problem that accumulation in SLN by administration of ICG alone is low. It is believed that low accumulation in SLN causes weak signals in detection of SLN by photoacoustic imaging. The reason of the low accumulation in SLN is believed that ICG is a low-molecular compound and is therefore rapidly excreted to the outside of the body. Accordingly, aspects of the present invention provide a photoacoustic imaging contrast agent that is highly accumulated in SLN.

The particles according to a first aspect of the present invention include a coloring agent having an anionic functional group, ions of a polyvalent metal, and a polyol compound.

The particles according to a second aspect of the present invention include a coloring agent having an anionic functional group, ions of a polyvalent metal, and a hydrophobic polymer.

The photoacoustic imaging contrast agent according to a third aspect of the present invention includes a coloring agent having an anionic functional group and ions of a polyvalent metal.

The SLN contrast agent according to a fourth aspect of the present invention includes a coloring agent having an anionic functional group and ions of a polyvalent metal.

Aspects of the present invention can provide particles, a photoacoustic imaging contrast agent, and an SLN contrast agent, where a hydrophilic coloring agent having an anionic functional group, such as ICG, hardly leaks from the particles.

Aspects of the present invention can provide an SLN contrast agent and a photoacoustic imaging contrast agent that highly accumulate in SLN to emit strong detection signals.

Further features of the present invention will become apparent from the following description of exemplary embodiments with reference to the attached drawings.

### BRIEF DESCRIPTION OF THE DRAWINGS

FIGS. 1A and 1B are diagrams illustrating particles according to a first embodiment of the present invention.

FIGS. 2A and 2B are diagrams illustrating particles according to a second embodiment of the present invention.

FIGS. 3A and 3B are diagrams illustrating particles according to a fourth embodiment of the present invention.

### DESCRIPTION OF THE EMBODIMENTS

First to fourth embodiments of the present invention will now be described, but the present invention is not limited thereto.

First of all, factors common to the first to fourth embodiments will be described.

### Coloring Agent Having an Anionic Functional Group

In the embodiments, the coloring agent having an anionic functional group is not particularly limited, but, for example, a coloring agent having a molar absorbance coefficient of 10⁶ M⁻¹-cm⁻¹ or more at least at a wavelength selected from the range of 600 to 1300 nm can be used.

Examples of the coloring agent having an anionic functional group include azine, acridine, triphenylmethane, xanthene, porphyrin, cyanine, phthalocyanine, styryl, pylyrium, azo, quinone, tetracycline, flavone, polyene, BODIPY (registered trademark), indigoid, and tar dyes each having an anionic functional group. The coloring agent having an anionic functional group may be a sodium salt or a potassium salt.

Examples of the cyanine dye include ICG, Alexa Fluor (registered trademark) dyes (manufactured by Invitrogen), Cy (registered trademark) dyes (manufactured by GE Healthcare Bioscience), IR-783, IR-806, and IR-820 (manufactured by Sigma-Aldrich Japan), IRDye 800CW and IRDye 800RS (registered trademark) (manufactured by LI-COR), ADS780WS, ADS795WS, ADS830WS, and ADS832WS (manufactured by American Dye Source, Inc.). Examples of the indigoid dye include indigo carmine. Examples of the tar dye include Patent Blue.

The above-mentioned coloring agents having an anionic functional group may be contained alone or in combination in the particles, photoacoustic imaging contrast agent, or SLN contrast agent according to the embodiments.

### Polyvalent Metal Ions

The polyvalent metal ions in the embodiments may be any di- or polyvalent metal ions.

When the source material of the coloring agent having an anionic functional group is, for example, a sodium salt or a potassium salt, ions of a polyvalent metal having an ionization tendency lower than that of the salt of such as sodium or potassium can be used. The use of ions of a metal having a lower ionization tendency accelerates exchange of metal ions to easily cause hydrophobization of the coloring agent having an anionic functional group.

Examples of the polyvalent metal ion in the embodiments include iron ions (Fe²⁺ and Fe³⁺), zinc ions, tin ions, manganese ions, magnesium ions, calcium ions, and aluminum ions. It is believed that a trivalent metal ion can bond to the coloring agent having an anionic functional group at multiple points to further stabilize a particle. Accordingly, iron ions (Fe³⁺) and aluminum ions can be particularly used as the polyvalent metal ions.

The polyvalent metal ions contained in the particles, photoacoustic imaging contrast agent, or SLN contrast agent according to the embodiments may be those of one polyvalent metal or of two or more polyvalent metals.

In preparation of the particles, photoacoustic imaging contrast agent, or SLN contrast agent that includes iron ions, for example, iron(III) chloride or iron(II) chloride can be used as a metal-containing reagent.

In preparation of the particles, photoacoustic imaging contrast agent, or SLN contrast agent that includes zinc ions, for example, zinc chloride or zinc acetate can be used as a metal-containing reagent.

In preparation of the particles, photoacoustic imaging contrast agent, or SLN contrast agent that includes tin ions, for example, tin chloride can be used as a metal-containing reagent.

In preparation of the particles, photoacoustic imaging contrast agent, or SLN contrast agent that includes manganese ions, for example, manganese chloride can be used as a metal-containing reagent.

In preparation of the particles, photoacoustic imaging contrast agent, or SLN contrast agent that includes magnesium ions, for example, magnesium chloride can be used as a metal-containing reagent.

In preparation of the particles, photoacoustic imaging contrast agent, or SLN contrast agent that includes calcium ions, for example, calcium chloride can be used as a metal-containing reagent.

In preparation of the particles, photoacoustic imaging contrast agent, or SLN contrast agent that includes aluminum ions, for example, aluminum chloride can be used as a metal-containing reagent.

These metal-containing reagents may be used alone or in any mixture thereof.

### Solvent Used in Production

The particles, photoacoustic imaging contrast agent, and SLN contrast agent according to the embodiments can be produced by various processes.

Examples of the solvent used in the production process include organic solvents and aqueous solvents. Examples of the organic solvents include hydrocarbons such as hexane, cyclohexane, and heptane; ketones such as acetone and methyl ethyl ketone; ethers such as tetrahydrofuran and diethyl ether; halogenated hydrocarbons such as dichloromethane, chloroform, carbon tetrachloride, dichloroethane, and trichloroethane; aromatic hydrocarbons such as benzene and toluene; esters such as ethyl acetate and butyl acetate; aprotic polar solvents such as N,N-dimethylformamide and dimethyl sulfoxide; and pyridine derivatives. Examples of the aqueous solvents include buffer solutions such as PBS, HEPES; and water. These solvents may be used alone or in any mixture thereof.

Distillation of Organic Solvent from Particle Dispersion

In the case of using an organic solvent in the production process, the organic solvent can be distilled away from a particle dispersion.

The distillation can be performed by any known method, and examples of the method include a removing method by heating and a method using a decompression device such as an evaporator.

The removing method by heating can be performed at any heating temperature that can prevent high-order aggregation that causes a reduction in yield of particles, and can be performed, for example, at a temperature range of 0° C. or more and 80° C. or less.

The distillation is not limited to the above-mentioned methods as long as aspects of the present invention can be achieved.

### Purification of Particle Dispersion

The prepared particle dispersion can be purified by any known method, and examples of the method include size exclusion column chromatography, ultrafiltration, dialysis, and centrifugation.

The purification is not limited to the above-mentioned methods as long as aspects of the present invention can be achieved.

### Particle Shape and Size

The particles, photoacoustic imaging contrast agent, and SLN contrast agent according to the embodiments may have any shape as long as they are solids, i.e., particles formed by aggregation of the coloring agent having an anionic functional group and ions of the polyvalent metal, for example, in a spherical, elliptic, planar, or one-dimensional string form.

The particles, photoacoustic imaging contrast agent, and SLN contrast agent according to the embodiments may have any size (particle diameter) such as 1 nm or more and 1000 nm or less, in particular, from 10 to 500 nm. A size of 1000 nm or less hardly causes formation of a blood clot in a vessel.

Each embodiment will be described below.

### First Embodiment

The particles according to a first embodiment of the present invention include a coloring agent having an anionic functional group, ions of a polyvalent metal, and a polyol compound.

Throughout the specification, the term “polyol compound” means a compound having two or more hydroxyl groups.

The coloring agent having an anionic functional group and the polyvalent metal ions are bound to each other by ionic bonds or coordinate bonds, and the polyvalent metal ions and the polyol compound are bound to each other by ionic bonds or coordinate bonds to form particles. The formation of such particles neutralizes the negative charge of the coloring agent having an anionic functional group with a part of the positive charge of the polyvalent metal ions. The residual positive charge of the polyvalent metal ions is partially neutralized by negative charge generated by dissociation of hydrogen atoms from the hydroxyl groups of the polyol compound. Thus, it is believed that in the particles according to the first embodiment, the hydrophobicity of the coloring agent having an anionic functional group is increased by neutralization of the negative charge of the anionic functional group of the coloring agent to reduce the affinity to water. Consequently, in the particles according to the first embodiment, the coloring agent having an anionic functional group hardly leaks.

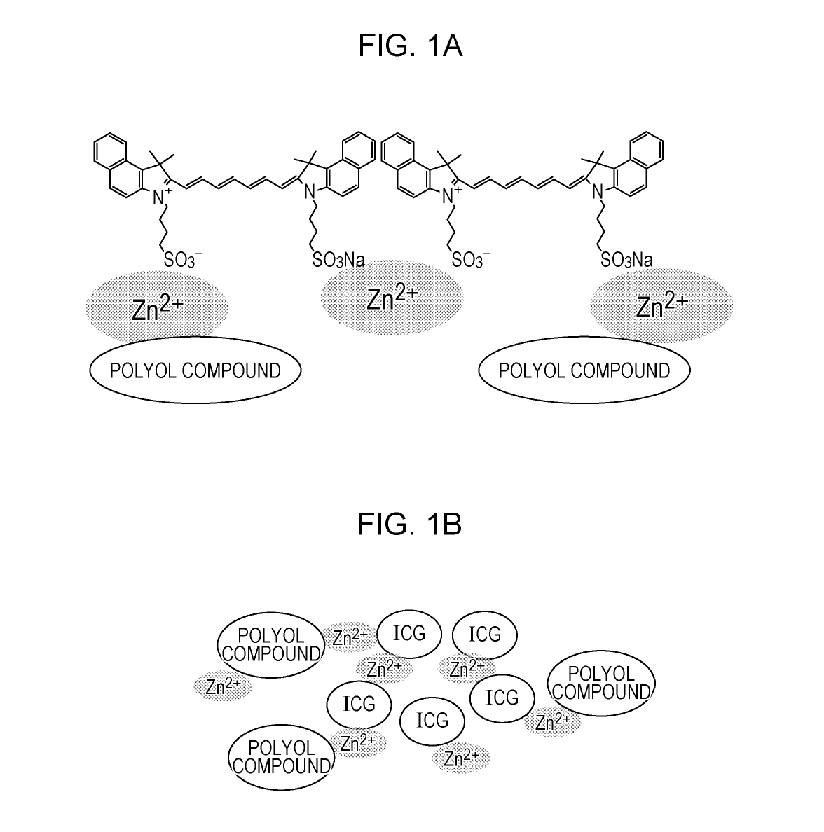

The mechanism of forming the above-described particles will be described with reference to FIGS. 1A and 1B. FIGS. 1A and 1B show an example of using ICG as the coloring agent having an anionic functional group and a zinc(II) ion (Zn²⁺) as the polyvalent metal ion. A zinc(II) ion has a divalent positive charge and is attracted to the negative charge of the two sulfonate groups (anionic functional groups) of ICG to form an ionic bond. As shown in FIGS. 1A and 1B, in some zinc(II) ions, one zinc(II) ion binds to one ICG, and in other some zinc(II) ions, one zinc(II) ion binds to two sulfonate groups from different ICG molecules, resulting in approaching of the ICG molecules to each other (FIG. 1A). The approaching of the ICG molecules increases the hydrophobicity thereof to enhance gathering of other ICG molecules binding or not binding to zinc(II) ions (FIG. 1B). Thus, these ICG molecules binding or not binding to zinc(II) ions gather to form particles. Furthermore, a part of the polyvalent metal ions bound to the coloring agent having an anionic functional group by ionic bonds or coordinate bonds have excess positive charge, and this positive charge attracts negative charge generated by dissociation of hydrogen atoms from the hydroxyl groups of the polyol compound to form ionic bonds or coordinate bonds. Thus, particles having higher stability are formed. The presence of the polyol compound allows formation of particles with higher stability, so that the particles have a structure where the coloring agent having an anionic functional group hardly migrates to the aqueous phase, i.e., the coloring agent hardly leaks from the particles.

The presence of ionic bond or coordinate bond can be confirmed by a shift of a peak in the spectrum obtained by infrared spectroscopy (IR).

The particles according to the first embodiment may include one or more coloring agents having an anionic functional group, may include ions of one or more polyvalent metals, and may include one or more polyol compounds.

### Polyol Compound

The polyol compound in the first embodiment may be any compound having two or more hydroxyl groups.

The polyol compound in the first embodiment can have an average molecular weight of 500 or more and 1000000 or less, in particular, 1000 or more and 600000 or less.

Examples of the polyol compound in the first embodiment include dextran (Chemical Formula (2)), heparin (Chemical Formula (3)), polysaccharides such as pullulan, polyphenols such as tannic acid, and derivatives thereof.

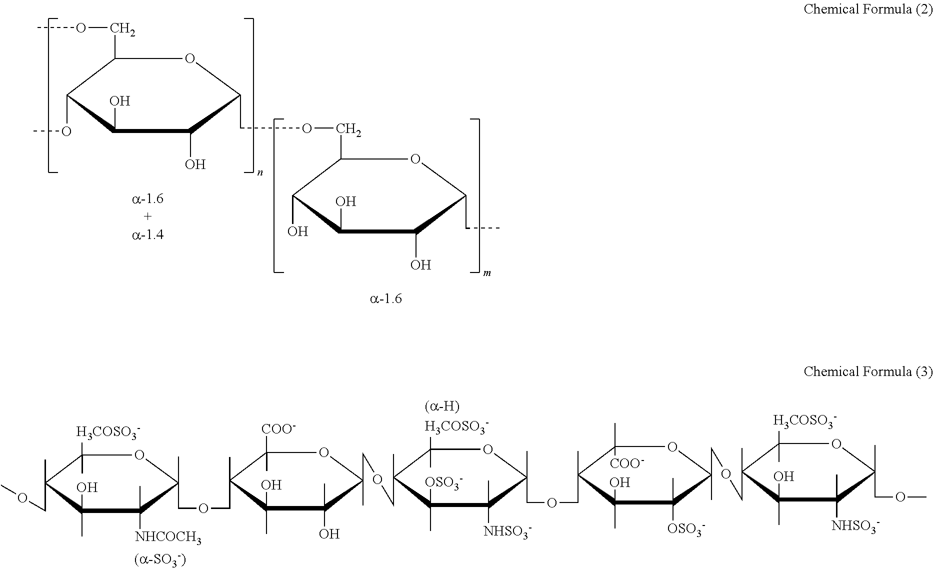

### Ratio of Constitutional Components of Particles

In the first embodiment, the content of the polyvalent metal ions can be 0.01% by weight or more and 20.0% by weight or less, in particular, 0.1% by weight or more and 10.0% by weight or less, based on 100% by weight of the particles. It is believed that when the content of the polyvalent metal ions is 20.0% by weight or less, the biotoxicity is low.

In the first embodiment, the content of the polyol compound can be 1.0% by weight or more and 75.0% by weight or less, in particular, 5.0% by weight or more and 50.0% by weight or less, based on 100% by weight of the particles. It is believed that when the content of the polyol compound is 1.0% by weight or more, the particles can have sufficient stability in vivo.

### Process of Producing Particles

The particles according to the first embodiment can be produced using a known process. For example, a polyol compound, a coloring agent having an anionic functional group, and ions of a polyvalent metal are mixed in a solvent to precipitate particles.

In this process, particles can be prepared by a known stirring method.

Compositional ratio of each material in the production process

In the above-described process of producing the particles, the volume ratio of a coloring agent having an anionic functional group solution (2) to a polyvalent metal ion solution (3) is not particularly limited in a range in which the particles can be yielded, and can be, for example, 0.001:1 to 1:1000.

The concentration of the coloring agent having an anionic functional group is not particularly limited in a range in which the coloring agent having an anionic functional group can be dissolved, and can be, for example, 0.0005 to 300 mg/ml.

The molar ratio of the coloring agent solution (3) can be 0.01 equivalents or more and 100 equivalents or less, in particular, 0.1 equivalents or more and 10 equivalents or less, with respect to the metal ion solution (2).

In the above-described process of producing the particles, the volume ratio of a polyol compound solution (1) to a coloring agent having an anionic functional group solution (2) is not particularly limited in a range in which the particles can be yielded, and can be, for example, 0.001:1 to 1:1000.

The volume ratio of a polyvalent metal ion solution (3) to a coloring agent having an anionic functional group solution (2) is not particularly limited in a range in which the particles can be yielded, and can be, for example, 0.001:1 to 1:1000.

The concentration of the coloring agent having an anionic functional group is not particularly limited in a range in which the coloring agent having an anionic functional group can be dissolved, and can be, for example, 0.0005 to 300 mg/ml.

The molar ratio of the polyol compound solution (1) or the metal ion solution (3) can be 0.01 equivalents or more and 100 equivalents or less, in particular, 0.1 equivalents or more and 10 equivalents or less, with respect to the coloring agent solution (2).

### Second Embodiment

The particles according to a second embodiment of the present invention include a coloring agent having an anionic functional group, ions of a polyvalent metal, and a hydrophobic polymer.

The coloring agent having an anionic functional group and the polyvalent metal ions form ionic bonds, and the ionic bond product of the coloring agent and the metal ions is covered by the hydrophobic polymer to form particles. Thus, it is believed that the hydrophobicity of the coloring agent having an anionic functional group is increased by neutralization of the negative charge of the anionic functional group of the coloring agent with a part of the positive charge of the polyvalent metal ions to reduce the affinity of the coloring agent to water. Consequently, in the particles according to the second embodiment, the coloring agent having an anionic functional group hardly leaks. In addition, since the hydrophobic polymer covers the coloring agent and the metal ions, in the particles according to the second embodiment, the coloring agent having an anionic functional group hardly leaks.

It is believed that the coloring agent having an anionic functional group and the polyvalent metal ions bound by ionic bonds, i.e., the aggregate of the coloring agent having an anionic functional group and the polyvalent metal ions is covered by the hydrophobic polymer to form particles, but a part of the coloring agent and the polyvalent metal ions exist on the surfaces of the particles.

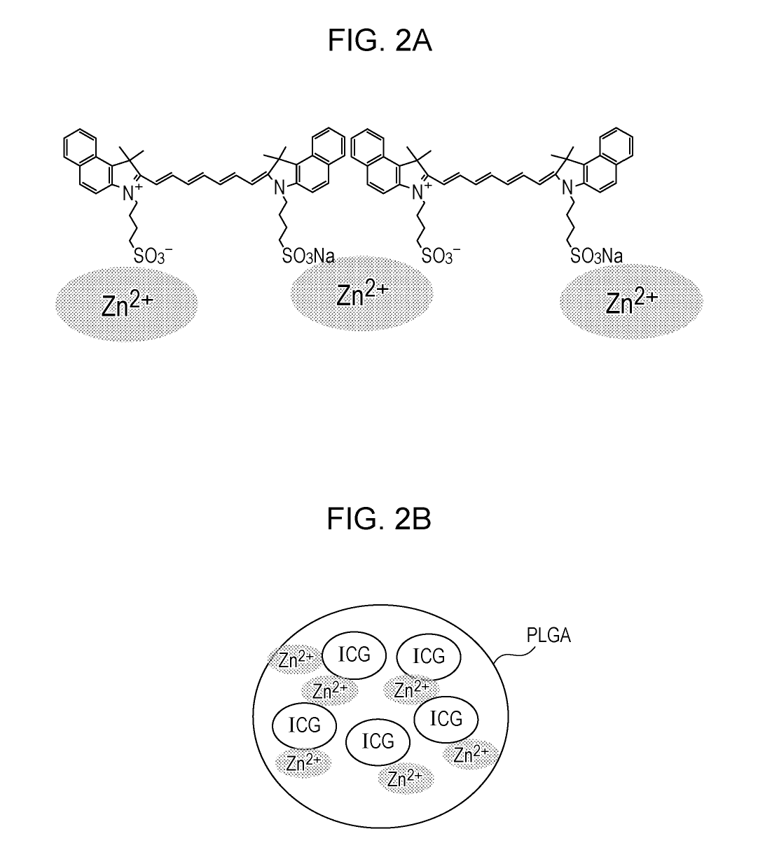

The mechanism of forming the above-described particles will be described with reference to FIGS. 2A and 2B. FIGS. 2A and 2B show an example of using ICG as the coloring agent having an anionic functional group, a zinc(II) ion (Zn²⁺) as the polyvalent metal ion, and PLGA as the hydrophobic polymer. A zinc(II) ion has a divalent positive charge and is attracted to the negative charge of the two sulfonate groups of ICG to form an ionic bond. As shown in FIGS. 2A and 2B, in some zinc(II) ions, one zinc(II) ion binds to one ICG, and in other some zinc(II) ions, one zinc(II) ion binds to two sulfonate groups from different ICG molecules, resulting in approaching of the ICG molecules to each other (FIG. 2A). The approaching of the ICG molecules increases the hydrophobicity thereof to enhance gathering of ICG molecules binding or not binding to zinc(II) ions (FIG. 2B). Thus, these ICG molecules binding or not binding to zinc(II) ions gather to form particles. In addition, since PLGA covers aggregates of the ionic bond product of ICG and polyvalent metal ions, free ICG, and free polyvalent metal ions, the coloring agent having sulfonate groups is prevented from coming into contact with water and therefore hardly migrates to the aqueous phase.

The presence of ionic bond or coordinate bond can be confirmed by a shift of a peak in the spectrum obtained by IR.

The particles according to the second embodiment may include one or more coloring agents having an anionic functional group, may include ions of one or more polyvalent metals, and may include one or more hydrophobic polymers. The particles according to the second embodiment may include a dispersion stabilizer. The use of the dispersion stabilizer can prevent the particles from agglomerating in water.

### Hydrophobic Polymer

The hydrophobic polymer in the second embodiment may be any polymer that is insoluble or hardly soluble in water and does not swell in water. Examples of the hydrophobic polymer in the second embodiment include homopolymers of monomers selected from hydroxycarboxylic acids having six or less carbon atoms, copolymers of two or more of the monomers, PLGA, polystyrenes, polymethacrylic acids, and derivatives of these polymers.

These polymers can have an average molecular weight of 2000 or more and 1000000 or less, in particular, 10000 or more and 600000 or less.

These polymers may be used alone or in a mixture thereof.

### Dispersion Stabilizer

The dispersion stabilizer in the second embodiment may be a surfactant or a hydrophilic polymer.

Examples of the surfactant include partially hydrolyzed polyvinyl alcohol (Chemical Formula (4), hereinafter, may be abbreviated as PVA), polyoxyethylene alkyl ethers, alkyl sulfates, phospholipids, polyoxyethylene sorbitan fatty acid esters, and polyoxyethylene polyoxypropylene block copolymers.

Examples of the polyoxyethylene sorbitan fatty acid ester include Tween 20, Tween 40, Tween 60, and Tween 80.

Examples of the phospholipid include phosphatidyl phospholipids including a PEG chain and a functional group selected from amino, NHS, maleimide, and methoxy groups.

Examples of the phosphatidyl phospholipid include 3-(N-succinimidyloxyglutaryl) aminopropyl, polyethyleneglycol-carbamyl distearoylphosphatidyl-ethanolamine (DSPE-PEG-NHS), N-(3-maleimide-1-oxopropyl) aminopropyl polyethyleneglycol-carbamyl distearoylphosphatidyl-ethanolamine (DSPE-PEG-MAL), N-(aminopropyl polyethyleneglycol)-carbamyl distearoylphosphatidyl-ethanolamine (DSPE-PEG-NH2), N-(carbonyl-methoxypolyethyleneglycol 2000)-1,2-distearoyl-sn-glycero-3-phosphoethanolamine, sodium salt (SUNBRIGHT DSPE-020CN), and N-(carbonyl-methoxypolyethyleneglycol 5000)-1,2-distearoyl-sn-glycero-3-phosphoethanolamine, sodium salt (SUNBRIGHT DSPE-050CN).

Examples of the polyoxyethylene polyoxypropylene block copolymer include compounds represented by Chemical Formula (5). In Chemical Formula (5), x and z are each independently an integer of 70 to 110, in particular, 75 to 106. Furthermore, in Chemical Formula (5), y is an integer of 20 to 80, in particular, 30 to 70.

Examples of the compounds represented by Chemical Formula (5) having x and z each being 75 and y being 30 include Pluronic (registered trademark) F68 (Chemical Formula (5)). Examples of the compounds represented by Chemical Formula (5) having x and z each being 106 and y being 70 include Pluronic (registered trademark) F127.

Examples of the hydrophilic polymer include polysaccharides such as dextran (Chemical Formula (2)), starch, and heparin (Chemical Formula (3)). Furthermore, sodium heparin, which is a sodium salt of heparin can be used.

These dispersion stabilizers may be used alone or in any mixture thereof.

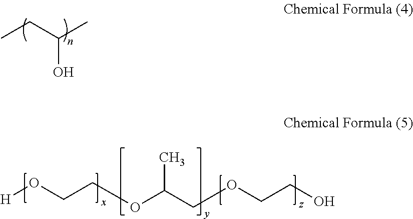

### Process of Producing Particles

The process of producing the particles of the second embodiment includes a step of preparing a mixture A of a coloring agent having an anionic functional group, a polyvalent metal ion salt, a hydrophobic polymer, and an organic solvent; and a step of mixing the mixture A and water, wherein the organic solvent is dimethyl sulfoxide (hereinafter, may be abbreviated as DMSO).

In one known method of producing particles, a mixture of a coloring agent having an anionic functional group and an organic solvent is dispersed in water to prepare particles.

In this method, however, the organic solvent for dissolving the coloring agent having an anionic functional group must be mixable with water, such as methanol.

Consequently, the method has a problem that the coloring agent having an anionic functional group dissolved in a solvent such as methanol migrates to the aqueous phase when the solvent is dispersed in water.

In the process of producing particles of the second embodiment, the coloring agent having an anionic functional group and the polyvalent metal ions are dissolved in DMSO to form coordinate bonds or ionic bonds in the DMSO.

In another known method of producing particles, the particles are prepared by converting a reagent having an anionic functional group into a water-insoluble salt with zinc acetate and then dissolving the reagent in acetone.

This method, however, cannot be applied to coloring agents insoluble in acetone, such as ICG.

The process of producing particles of the second embodiment can prepare particles without using acetone and therefore can be applied to coloring agents such as ICG.

In an example of the process of producing particles according to the second embodiment, the particles are prepared by dispersing a mixture of a coloring agent having an anionic functional group, a polyvalent metal ion salt, a hydrophobic polymer, and an organic solvent in a dispersion medium including water as a main component; and distilling away the organic solvent dissolved/dispersed in the dispersion medium including water as a main component by, for example, evaporation. In order to impart high dispersion stability to the finally resulting particles, the mixture may be mixed with a dispersion medium containing a dispersion stabilizer.

Specifically, a known producing method, such as nanoemulsification or nanoprecipitation, can be employed.

In the nanoemulsification, an emulsion can be prepared by known emulsification. Examples of the known emulsification include intermittent vibration, stirring using a mixer such as a propeller agitator or a turbine agitator, a colloid mill method, a homogenizer method, and ultrasonic wave irradiation. These methods may be employed alone or in combination thereof. The emulsion may be prepared by one-step emulsification or by multi-step emulsification. The emulsification is not limited thereto as long as aspects of the present invention can be achieved.

In the nanoprecipitation, particles can be prepared by a known stirring method.

### Compositional Ratio of Each Material in the Production Process

In the nanoemulsification, the weight ratio of a dispersion-stabilizer aqueous solution to the organic solvent is not particularly limited in a range in which an oil-in-water (O/W) emulsion can be formed. The weight ratio of the organic solvent to the aqueous solution can be 1:2 to 1:1000.

In the nanoprecipitation, the weight ratio of the dispersion-stabilizer aqueous solution to the organic solvent is not particularly limited in a range in which the particles can be yielded. The weight ratio of the organic solvent to the dispersion-stabilizer aqueous solution can be 1:1 to 1:1000.

The concentrations of the hydrophobic polymer and the coloring agent having an anionic functional group in an organic solvent are not particularly limited in a ranges in which they can be dissolved in the solvent. For example, the concentration of the hydrophobic polymer can be 0.3 mg/mL or more and 100 mg/mL or less; and the concentration of the coloring agent having an anionic functional group can be 0.0005 mg/mL or more and 300 mg/mL or less.

The weight ratio of the coloring agent having an anionic functional group to the hydrophobic polymer in the organic solvent can be 100:1 to 100:1000.

The concentration of the polyvalent metal ion salt in an organic solvent is not particularly limited in a range in which it can be dissolved in the solvent. For example, the concentration can be 0.001 mg/mL or more and 100 mg/mL or less. In addition, the weight ratio of the hydrophilic coloring agent to the metal ions in the organic solvent can be in the range of 100:1 to 1:1000.

### Dispersion-Stabilizer Aqueous Solution

The concentration of the dispersion stabilizer contained in the dispersion-stabilizer aqueous solution is not particularly limited in a range in which it can be dissolved in the solution. For example, the content of the polyoxyethylene polyoxypropylene block copolymer contained in the aqueous solution can be 0.01 mg/mL or more and 300 mg/mL or less.

### Third Embodiment

The photoacoustic imaging contrast agent according to a third embodiment of the present invention includes a coloring agent having an anionic functional group and ions of a polyvalent metal.

In the photoacoustic imaging contrast agent according to the third embodiment, a coloring agent having an anionic functional group and ions of a polyvalent metal are bound to each other by ionic bonds or coordinate bonds to form particles. The resulting particles have a larger particle diameter than that of a low molecular compound.

The photoacoustic imaging contrast agent according to the third embodiment may contain one or more coloring agents having an anionic functional group.

The photoacoustic imaging contrast agent according to the third embodiment may contain ions of one or more polyvalent metals.

The photoacoustic imaging contrast agent according to the third embodiment can further include a polyol compound. The polyol compound binds to the polyvalent metal ions to form particles. The photoacoustic imaging contrast agent in the third embodiment may contain one or more polyol compounds.

The presence of ionic bond or coordinate bond can be confirmed by a shift of a peak in the spectrum obtained by IR.

### Ratio of Constitutional Components of Particles for Photoacoustic Imaging Contrast Agent

In the particles according to the third embodiment, the content of the polyvalent metal ions can be 0.01% by weight or more and 20.0% by weight or less, in particular, 0.1% by weight or more and 10.0% by weight or less, based on 100% by weight of the particles.

It is believed that when the content of the polyvalent metal ions is 20.0% by weight or less, the biotoxicity is low.

When the particles according to the third embodiment include a polyol compound, the content of the polyol compound can be 1.0% by weight or more and 75.0% by weight or less, in particular, 5.0% by weight or more and 50.0% by weight or less based on 100% by weight of the particles. It is believed that when the content of the polyol compound is 1.0% by weight or more, the particles are sufficiently stable in vivo.

### Process of Producing Particles for Photoacoustic Imaging Contrast Agent

The particles according to the third embodiment can be produced using a known process. For example, a coloring agent having an anionic functional group and ions of a polyvalent metal are mixed in a solvent to precipitate particles.

In this process, particles can be prepared by a known stirring method.

In the above-described process of producing the particles, the volume ratio of a coloring agent having an anionic functional group solution (2) to a polyvalent metal ion solution (3) is not particularly limited in a range in which the particles can be yielded, and can be in the range of 0.001:1 to 1:1000.

The concentration of the coloring agent having an anionic functional group is not particularly limited in a range in which the coloring agent can be dissolved in the solvent. For example, the concentration of the coloring agent having an anionic functional group can be 0.0005 mg/mL or more and 300 mg/mL or less.

The molar ratio of the metal ion solution (3) can be 0.01 equivalents or more and 100 equivalents or less, in particular, 0.1 equivalents or more and 10 equivalents or less with respect to the coloring agent solution (2).

The volume ratio of a polyvalent metal ion solution (3) to a coloring agent having an anionic functional group solution (2) is not particularly limited in a range in which the particles can be yielded, and can be in the range of 0.001:1 to 1:1000.

The concentration of the coloring agent having an anionic functional group is not particularly limited in a range in which it can be dissolved in the solvent. For example, the concentration of the coloring agent having an anionic functional group can be 0.0005 mg/mL or more and 300 mg/mL or less.

When the particles include a polyol compound, the particles are produced under the same conditions as in the first embodiment and can be used as particles for a photoacoustic imaging contrast agent.

### Fourth Embodiment

The SLN contrast agent according to a fourth embodiment of the present invention includes a coloring agent having an anionic functional group and ions of a polyvalent metal.

In the SLN contrast agent according to the fourth embodiment, a coloring agent having an anionic functional group and ions of a polyvalent metal are bound to each other by ionic bonds or coordinate bonds to form particles. The resulting particles have a larger particle diameter than that of a low molecular compound. The migration velocity of the particles in a lymph vessel is reduced with an increase in particle diameter. It is therefore believed that the migration velocity of the particles to lymph nodes on the downstream side of SLN is reduced to increase the amount of the particles accumulated in SLN compared with the case of using a low molecular weight compound alone. An increase in amount of the particles accumulating in SLN increases the photoacoustic signals obtained from the SLN. Imaging by a photoacoustic imaging method can therefore provide a clear image of the SLN.

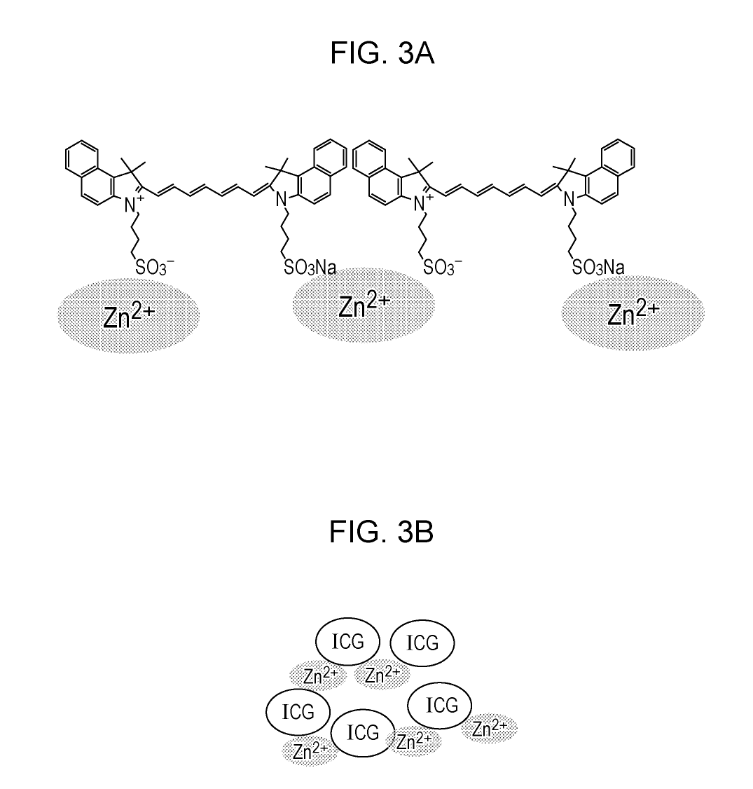

The mechanism of forming the above-described particles will be described with reference to FIGS. 3A and 3B. FIGS. 3A and 3B show an example of using ICG as the coloring agent having an anionic functional group and a zinc(II) ion (Zn²⁺) as the polyvalent metal ion. A zinc(II) ion has a divalent positive charge and is attracted to the negative charge of the two sulfonate groups of ICG to form an ionic bond. As shown in FIGS. 3A and 3B, in some zinc(II) ions, one zinc(II) ion binds to one ICG, and in other some zinc(II) ions, one zinc(II) ion binds to two sulfonate groups from different ICG molecules, resulting in approaching of the ICG molecules to each other (FIG. 3A). The approaching of the ICG molecules increases the hydrophobicity thereof to enhance gathering of ICG molecules binding or not binding to zinc(II) ions (FIG. 3B). Thus, ICG molecules binding or not binding to zinc(II) ions gather to form particles.

The presence of ionic bond or coordinate bond can be confirmed by a shift of a peak in the spectrum obtained by IR.

The SLN contrast agent according to the fourth embodiment highly accumulates in SLN as shown in Examples described below and can be therefore applied to imaging of SLN. The SLN contrast agent according to the fourth embodiment may further include a polyol compound. A part of the polyvalent metal ions bound to the coloring agent having an anionic functional group by ionic bonds or coordinate bonds have excess positive charge, and this positive charge attracts negative charge generated by dissociation of hydrogen atoms from the hydroxyl groups of the polyol compound to form ionic bonds or coordinate bonds. Thus, particles having higher stability are formed.

That is, the presence of the polyol compound allows formation of higher stable particles having a structure where the coloring agent hardly migrates to the aqueous phase.

The photoacoustic imaging contrast agent and the photoacoustic imaging will now be described.

### Photoacoustic Imaging Contrast Agent

The photoacoustic imaging contrast agent according to the fourth embodiment includes the particles according to any one of the above-described embodiments and a dispersion medium. The dispersion medium is a liquid material, and examples thereof include physiological saline solutions, distilled water for injection, and phosphate buffered saline (hereinafter, may be abbreviated as PBS). The photoacoustic imaging contrast agent according to the fourth embodiment optionally includes pharmacologically acceptable additives.

In the photoacoustic imaging contrast agent according to the fourth embodiment, the particles may be dispersed in the dispersion medium in advance, or the particles according to any one of the above-described embodiments may be prepared as a kit and be used by dispersing the particles in the dispersion medium prior to administration in vivo.

### Photoacoustic Imaging

The photoacoustic imaging contrast agent according to the fourth embodiment can be used in photoacoustic imaging. Throughout the specification, the photoacoustic imaging is a concept including photoacoustic tomography. The photoacoustic imaging using the photoacoustic imaging contrast agent according to the fourth embodiment includes at least a step of administering the photoacoustic imaging contrast agent according to the fourth embodiment to a subject or adding the agent to a sample obtained from the subject; a step of irradiating the subject or the sample with pulsed light; and a step of measuring photoacoustic signals derived from the particles present in the subject or the sample.

An example of the photoacoustic imaging using the photoacoustic imaging contrast agent according to the fourth embodiment is as follows: the photoacoustic imaging contrast agent according to the fourth embodiment is administered to a subject or is added to a sample, such as an organ, obtained from the subject. Examples of the subject include mammals such as human, experimental animals, and pets, but the subject is not limited thereto and may be any organism. Examples of the sample obtained from a subject include organs, tissues, tissue sections, cells, and cell lysates. After administration or addition of the particles, the subject or the sample is irradiated with near-infrared laser pulses.

In the photoacoustic imaging according to the fourth embodiment, the wavelength of light for irradiation can be selected by selecting the laser source used. In the photoacoustic imaging according to the fourth embodiment, in order to efficiently obtain acoustic signals, a subject or a sample may be irradiated with light having a wavelength in the near-infrared region of 600 to 1300 nm, which rarely affects absorption and diffusion of light in vivo and is called “biological window”.

A photoacoustic signal (acoustic wave) from the photoacoustic imaging contrast agent according to the fourth embodiment is detected with an acoustic wave detector, for example, a piezoelectric transducer, and is converted into an electric signal. Based on the electric signal detected with the acoustic wave detector, the location and size of an absorber in the subject or the sample and the distribution of optical characteristic values such as molar absorption coefficients can be calculated. For example, if the photoacoustic imaging contrast agent is detected at a value equal to or higher than a reference threshold, the subject can be presumed to have a target molecule or a site producing the target molecule; the sample can be presumed to have the target molecule; or the subject from which the sample is derived can be presumed to have a site producing the target molecule.

### EXAMPLES

In order to further clarify the features of the present invention, aspects of the present invention will now be described along Examples, but is not limited to these Examples, and the materials, compositional conditions, reaction conditions, and other factors can be freely changed within the ranges that a photoacoustic imaging contrast agent showing similar functions and effects can be obtained.

### Recovery Process

Centrifugation was performed with a high speed refrigerated microcentrifuge (MX-300, manufactured by Tomy Seiko Co., Ltd.). Ultracentrifugation was performed with a micro-ultracentrifuge (CS150GXL, manufactured by Hitachi Koki Co., Ltd.).

### Analysis Process

Particle sizes were measured with a dynamic light scattering analyzer (ELSZ-2, manufactured by Otsuka Electronics Co., Ltd.). A semiconductor laser was used as a light source, and the cumulant diameter was employed as a particle diameter.

Absorbance was measured with a UV-VIS-NIR spectrophotometer (Lambda Bio 40, manufactured by Perkin Elmer).

NMR measurement was performed with AVANCE 500 (manufactured by Bruker) under resonance frequency: 500 MHz, measurement nuclide: 1H, measurement temperature: room temperature, and solvent: heavy water.

Metal ions were measured with an inductively coupled plasma atomic emission spectrometer (ICP-AES) (CIROS, manufactured by SPECTRO).

### Photoacoustic Characteristics Evaluation Example

Photoacoustic signals were measured by irradiating a sample with pulsed laser light, detecting photoacoustic signals from the sample with a piezoelectric device, amplifying the signals with a high-speed preamplifier, and then obtaining the amplified signals with a digital oscilloscope. Specific conditions are as follows: A titanium-sapphire laser (manufactured by Lotis Ltd.) was used as a light source under conditions of a wavelength of 790 nm, an energy density of 12 mJ/cm², a pulse width of 20 nanoseconds, and a pulse repetition frequency of 10 Hz. An ultrasonic transducer (model: V303, manufactured by Panametrics-NDT) was used under conditions of a central frequency of 1 MHz, an element diameter of 0.5 mm, an object distance of 25 mm (non-focus), and an amplification of +30 dB (ultrasound preamplifier Model 5682, manufactured by Olympus Corp.). The measurement vessel was a poly(styrene) cuvette having an optical path length of 0.1 cm and a sample volume of approximately 200 μL. A measurement device, DPO4104 (manufactured by Tektronix), was used under conditions of a trigger: detection of photoacoustic light with a photodiode and data acquisition: average of 128 times (128 pulses).

### Calculation of Molar Absorption Coefficient Per Particle

(A) Solid weight of each constitutional component of a sample was calculated by NMR measurement, absorbance measurement, and metal ion quantitative determination.

(B) Weight of one particle was calculated from each particle diameter assuming that the density of each constitutional component was 1 g/cm³.

(C) Particle concentration of each sample solution was calculated from the results (A) and (B).

The molar absorption coefficient per particle was calculated from the results of absorbance measurement and the result (C).

### Evaluation of ICG-Retaining Performance of Particles in Fetal Bovine Serum (FBS)

The prepared particles were evaluated for performance of retaining coloring agent (e.g., ICG) using FBS (manufactured by Invitrogen). Two hundred microliters of a sample was dispersed in 1800 μL of FBS, and the resulting dispersion was left to stand at 36° C. for one day. The dispersion was ultracentrifuged at 288000 g, and the supernatant was recovered. The absorbance of the dispersion and the absorbance of the supernatant were measured. The remaining rate of coloring agent in the particles was calculated based on the following expression:

Remaining rate of coloring agent in particle=[1−(absorbance of supernatant)/(absorbance of dispersion)]×100.

### Evaluation of Accumulation in SLN

Migration to SLN was evaluated using mouse popliteal lymph nodes. A probe was administered to a mouse from the sole of the hindlimb, and accumulation in the popliteal lymph nodes was evaluated as a model of evaluation of accumulation in SLN. Ten microliters of a sample was subcutaneously administered to a mouse from the sole of the hindlimb, and after 24 hours, mouse popliteal lymph nodes were harvested. The harvested mouse popliteal lymph nodes were crushed, and the absorbance of the extract from the crushed lymph nodes was measured. The accumulation rate was calculated based on the following expression:

Accumulation rate=[(absorbance of the extracted mouse popliteal lymph nodes)/(absorbance of the sample in an amount equivalent to the administered amount)]×100.

### Example 1

In this Example, ICG was used as the coloring agent having an anionic functional group, and iron(III) chloride hexahydrate was used as a metal chloride.

ICG (7.0 mg, manufactured by Pharmaceutical and Medical Device Regulatory Science Society of Japan) was dissolved in 4.0 mL of a 10 mM 2-[4-(2-hydroxyethyl)-1-piperazinyl]ethanesulfonic acid buffer solution (hereinafter, may be simply abbreviated as HEPES) (manufactured by Invitrogen).

Iron(III) chloride hexahydrate (4.9 mg, manufactured by Wako Pure Chemical Industries, Ltd.), which was 2.0 equivalents with respect to ICG, was dissolved in 8.0 mL of 10 mM HEPES.

Tannic acid (13.0 mg, manufactured by Kishida Chemical Co., Ltd.), which was 1.0 equivalents with respect to ICG, was dissolved in 4.0 mL of 10 mM HEPES.

The ICG solution was added to the tannic acid solution with stirring, and the iron chloride solution was then added thereto to prepare a particle dispersion.

The resulting particle dispersion was centrifuged at 20000 g for 45 minutes at 4° C. to collect the particles.

The collected particles were washed with 10 mM HEPES, followed by centrifugation at 20000 g for 45 minutes at 4° C. to collect the particles. The collected particles were re-dispersed in 10 mM HEPES.

The resulting particle dispersion was filtered through a filter with a pore size of 0.45 μm to prepare Particle 1.

### Example 2

Particle 2 was prepared as in Example 1 except that iron(III) chloride hexahydrate was replaced by zinc(II) chloride (manufactured by Kishida Chemical Co., Ltd.).

### Example 3

Particle 3 was prepared as in Example 1 except that iron(III) chloride hexahydrate was replaced by aluminum(III) chloride (manufactured by Kishida Chemical Co., Ltd.).

### Example 4

Particle 4 was prepared as in Example 1 except that iron(III) chloride hexahydrate was replaced by magnesium(II) chloride hexahydrate (manufactured by Kishida Chemical Co., Ltd.).

### Example 5

Particle 5 was prepared as in Example 1 except that iron(III) chloride hexahydrate was replaced by calcium(II) chloride (manufactured by Nacalai Tesque, Inc.).

### Example 6

Particle 6 was prepared as in Example 1 except that iron(III) chloride hexahydrate was replaced by copper(II) chloride (manufactured by Kishida Chemical Co., Ltd.).

Table 1 shows the polyvalent metal ion used, maximum absorption wavelength, particle diameter, molar absorption coefficient per particle, and photoacoustic signal intensity per particle (wavelength: 790 nm) in the resulting Particles 1 to 6. The molar absorption coefficient per particle and the photoacoustic signal intensity per particle were calculated assuming that the particle diameter was 100 nm. Note that E+x in Table 1 represents 10 to the power of X. For example, 7.9E+10 means 7.9×10¹⁰. The same applies to the following tables.

TABLE 1

| | | | | | | | Molar | Photoacoustic | |
|---|---|---|---|---|---|---|---|---|---|
| | | | | Metal | Maximum | | absorption | signal | Remaining |
| | | | | chloride/ | absorption | Particle | coefficient | intensity | rate of ICG |
| | | | Polyol | ICG charging | wavelength | diameter | per particle | per particle | in particle |
| | | Metal chloride | compound | ratio | (nm) | (nm) | (M−1 cm−1) | (V J−1 M−1) | (%) |
| Example 1 | Particle 1 | Iron(III) ion | Tannic acid | 2.0 | 727 | 146 | 7.5E+10 | 3.1E+10 | 67 |
| Example 2 | Particle 2 | Zinc(II) ion | Tannic acid | 2.0 | 720 | 346 | 2.8E+10 | 4.1E+10 | 16 |
| Example 3 | Particle 3 | Aluminum(III) | Tannic acid | 2.0 | 720 | 214 | 7.5E+10 | 2.2E+10 | 56 |
| | | ion | | | | | | | |
| Example 4 | Particle 4 | Magnesium(II) | Tannic acid | 2.0 | 700 | 1024 | 3.1E+10 | 6.7E+10 | 7 |
| | | ion | | | | | | | |
| Example 5 | Particle 5 | Calcium(II) ion | Tannic acid | 2.0 | 700 | 825 | 3.2E+10 | 6.8E+10 | 6 |
| Example 6 | Particle 6 | Copper(II) ion | Tannic acid | 2.0 | 717 | 331 | 3.6E+10 | 8.8E+10 | 11 |

### Example 7

ICG (3.5 mg, manufactured by Pharmaceutical and Medical Device Regulatory Science Society of Japan) was dissolved in 2.0 mL of ultrapure water.

Iron(III) chloride hexahydrate (0.6 mg, manufactured by Wako Pure Chemical Industries, Ltd.), which was 0.5 equivalents with respect to ICG, was dissolved in 4.0 mL of ultrapure water.

Dextran (6.4 mg, manufactured by Tokyo Chemical Industry Co., Ltd.), which was 0.1 equivalents with respect to ICG, was dissolved in 2.0 mL of ultrapure water.

The ICG solution was added to the dextran solution with stirring, and the iron chloride solution was then added thereto to prepare a particle dispersion.

The resulting particle dispersion was centrifuged at 20000 g for 45 minutes at 4° C. to collect the particles. The collected particles were washed with ultrapure water, followed by centrifugation at 20000 g for 45 minutes at 4° C. to collect the particles. The collected particles were re-dispersed in ultrapure water.

The resulting particle dispersion was filtered through a filter with a pore size of 0.45 μm to prepare Particle 7.

### Example 8

Particle 8 was prepared as in Example 7 except that iron(III) chloride hexahydrate was replaced by zinc(II) chloride (manufactured by Kishida Chemical Co., Ltd.).

### Example 9

Particle 9 was prepared as in Example 7 except that iron(III) chloride hexahydrate was replaced by aluminum(III) chloride (manufactured by Kishida Chemical Co., Ltd.).

### Example 10

Particle 10 was prepared as in Example 7 except that iron(III) chloride hexahydrate was replaced by magnesium(II) chloride hexahydrate (manufactured by Kishida Chemical Co., Ltd.).

Table 2 shows the polyvalent metal ion used, maximum absorption wavelength, particle diameter, molar absorption coefficient per particle, and photoacoustic signal intensity per particle (wavelength: 790 nm) in the resulting Particles 7 to 10. The molar absorption coefficient per particle and the photoacoustic signal intensity per particle were calculated assuming that the particle diameter was 100 nm.

TABLE 2

| | | | | | | | Molar | Photoacoustic | |
|---|---|---|---|---|---|---|---|---|---|
| | | | | Metal | Maximum | | absorption | signal | Remaining |
| | | | | chloride/ | absorption | Particle | coefficient | intensity | rate of ICG |
| | | | Polyol | ICG charging | wavelength | diameter | per particle | per particle | in particle |
| | | Metal chloride | compound | ratio | (nm) | (nm) | (M−1 cm−1) | (V J−1 M−1) | (%) |
| Example 7 | Particle 7 | Iron(III) ion | Dextran | 0.5 | 779 | 55 | 5.4E+10 | 9.9E+10 | 12 |
| Example 8 | Particle 8 | Zinc(II) ion | Dextran | 0.5 | 779 | 177 | 3.9E+10 | 9.2E+10 | 11 |
| Example 9 | Particle 9 | Aluminum(III) | Dextran | 0.5 | 781 | 102 | 1.5E+10 | 6.4E+10 | 16 |
| | | ion | | | | | | | |
| Example 10 | Particle 10 | Magnesium(II) | Dextran | 0.5 | 778 | 410 | 4.0E+10 | 8.8E+10 | 10 |
| | | ion | | | | | | | |

### Example 11

ICG (5.5 mg, manufactured by Pharmaceutical and Medical Device Regulatory Science Society of Japan) and 2.0 mg of iron(III) chloride hexahydrate, which was 1.0 equivalents with respect to ICG, were dissolved in 2.0 mL of DMSO.

NaCl was added to 10 mM HEPES (manufactured by Invitrogen) to prepare a 0.3 M NaCl solution, and 60 mg of dextran (manufactured by Tokyo Chemical Industry Co., Ltd.) was dissolved in the solution.

The solution containing ICG and iron was added to the dextran solution with stirring to prepare a particle dispersion.

The resulting particle dispersion was centrifuged at 20000 g for 20 minutes at 4° C. to collect particles. The collected particles were re-dispersed in 10 mM HEPES.

The resulting particle dispersion was sequentially filtered through filters with pore sizes of 5.5, 1.2, 0.8, and 0.45 μm to prepare Particle 11.

### Example 12

Particle 12 was prepared as in Example 11 except that dextran (manufactured by Tokyo Chemical Industry Co., Ltd.) was replaced by sodium heparinate (Chemical Formula (6), manufactured by Tokyo Chemical Industry Co., Ltd.).

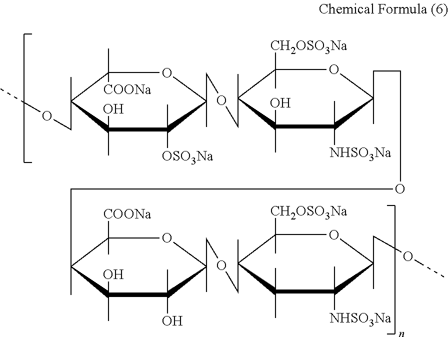

### Example 13

Particle 13 was prepared as in Example 11 except that iron(III) chloride hexahydrate was replaced by calcium(II) chloride (manufactured by Nacalai Tesque, Inc.).

### Example 14

Particle 14 was prepared as in Example 11 except that iron(III) chloride hexahydrate was replaced by magnesium(II) chloride hexahydrate (manufactured by Kishida Chemical Co., Ltd.).

### Example 15

Particle 15 was prepared as in Example 11 except that iron(III) chloride hexahydrate was replaced by manganese(II) chloride tetrahydrate.

### Example 16

Particle 16 was prepared as in Example 11 except that iron(III) chloride hexahydrate was replaced by tin(II) chloride dihydrate.

Table 3 shows the polyvalent metal ion used, maximum absorption wavelength, particle diameter, molar absorption coefficient per particle, and photoacoustic signal intensity per particle (wavelength: 790 nm) in Particles 11 to 16. The molar absorption coefficient per particle and the photoacoustic signal intensity per particle were calculated assuming that the particle diameter was 100 nm.

TABLE 3

| | | | | | | | Molar | Photoacoustic | |
|---|---|---|---|---|---|---|---|---|---|
| | | | | Metal | Maximum | | absorption | signal | Remaining |
| | | | | chloride/ | absorption | Particle | coefficient | intensity | rate of ICG |
| | | | Polyol | ICG charging | wavelength | diameter | per particle | per particle | in particle |
| | | Metal chloride | compound | ratio | (nm) | (nm) | (M−1 cm−1) | (V J−1 M−1) | (%) |
| Example 11 | Particle 11 | Iron(III) | Dextran | 1.0 | 785 | 149 | 4.5E+09 | 4.3E+10 | 46 |
| | | chloride | | | | | | | |
| Example 12 | Particle 12 | Iron(III) | Sodium | 1.0 | 781 | 130 | 4.3E+09 | 1.6E+10 | 31 |
| | | chloride | heparinate | | | | | | |
| Example 13 | Particle 13 | Calcium(II) | Dextran | 1.0 | 778 | 208 | 8.1E+09 | 1.3E+11 | 25 |
| | | chloride | | | | | | | |
| Example 14 | Particle 14 | Magnesium(II) | Dextran | 1.0 | 779 | 408 | 4.8E+10 | 8.3E+11 | 25 |
| | | chloride | | | | | | | |
| Example 15 | Particle 15 | Manganese(II) | Dextran | 1.0 | 778 | 361 | 3.6E+10 | 5.9E+11 | 48 |
| | | chloride | | | | | | | |
| Example 16 | Particle 16 | Tin(II) chloride | Dextran | 1.0 | 778 | 347 | 3.2E+10 | 5.1E+11 | 23 |

### Example 17

Particle 17 was prepared as in Example 1 except that ICG was replaced by indigo carmine (manufactured by Tokyo Chemical Industry Co., Ltd.).

### Example 18

Particle 18 was prepared as in Example 3 except that ICG was replaced by indigo carmine (manufactured by Tokyo Chemical Industry Co., Ltd.).

### Example 19

Particle 19 was prepared as in Example 1 except that ICG was replaced by Patent Blue (manufactured by Wako Pure Chemical Industries, Ltd.).

### Example 20

Particle 20 was prepared as in Example 3 except that ICG was replaced by Patent Blue (manufactured by Wako Pure Chemical Industries, Ltd.).

Table 4 shows the polyvalent metal ion used, maximum absorption wavelength, particle diameter, molar absorption coefficient per particle, and remaining rate of coloring agent in particles in Particles 17 to 20. The molar absorption coefficient per particle was calculated assuming that the particle diameter was 100 nm.

TABLE 4

| | | | | | | | | Molar | |
|---|---|---|---|---|---|---|---|---|---|
| | | Coloring agent | | | Metal | Maximum | | absorption | Remaining rate |
| | | having an | | | chloride/ | absorption | Particle | coefficient | of coloring |
| | | anionic | Polyvalent | Polyol | ICG charging | wavelength | diameter | per particle | agent in |
| | | functional group | metal ion | compound | ratio | (nm) | (nm) | (M−1 cm−1) | particle (%) |
| Example 17 | Particle 17 | Indigo | Iron(III) ion | Tannic acid | 2.0 | 605 | 85 | 6.5E+08 | 50 |
| | | carmine | | | | | | | |
| Example 18 | Particle 18 | Indigo | Aluminum(III) | Tannic acid | 2.0 | 610 | 105 | 1.8E+08 | 22 |
| | | carmine | ion | | | | | | |
| Example 19 | Particle 19 | Patent Blue | Iron(III) ion | Tannic acid | 2.0 | 639 | 81 | 1.0E+09 | 13 |
| Example 20 | Particle 20 | Patent Blue | Aluminum(III) | Tannic acid | 2.0 | 639 | 119 | 1.0E+09 | 7 |
| | | | ion | | | | | | |

### Example 21

A particle dispersion was prepared as in Example 1. In this Example, the resulting particle dispersion was centrifuged at 20000 g for 45 minutes at 4° C., and the resulting solution portion containing unprecipitated particles was ultracentrifuged at 72100 g for 15 minutes at 4° C. to obtain Particle 21 having a particle diameter further smaller than that of Particle 1 prepared in Example 1.

### Example 22

The solution portion containing unprecipitated particles in the ultracentrifugation in Example 21 was ultracentrifuged at 451000 g for 15 minutes at 4° C. to obtain Particle 22 having a particle diameter further smaller than that of Particle 21 prepared in Example 21.

Table 5 shows the coloring agent having an anionic functional group and polyvalent metal ion used, molar absorption coefficient per particle, and other factors in Particles 21 and 22. The molar absorption coefficient per particle was calculated assuming that the particle diameter was 100 nm.

TABLE 5

| | | | | | | | | Molar | |
|---|---|---|---|---|---|---|---|---|---|
| | | Coloring agent | | | Metal | Maximum | | absorption | Remaining rate |
| | | having an | | | chloride/ | absorption | Particle | coefficient | of coloring |
| | | anionic | Polyvalent | Polyol | ICG charging | wavelength | diameter | per particle | agent in |
| | | functional group | metal ion | compound | ratio | (nm) | (nm) | (M−1 cm−1) | particle (%) |
| Example 21 | Particle 21 | ICG | Iron(III) ion | Tannic acid | 2.0 | 798 | 80 | 4.5E+09 | — |
| Example 22 | Particle 22 | ICG | Iron(III) ion | Tannic acid | 2.0 | 796 | 49 | 4.6E+09 | — |

### Example A1

### Preparation of Organic Solvent Solution

An organic solvent solution was prepared by dissolving 5.5 mg of the Japanese pharmacopeia external standard indocyanine green standard (hereinafter, simply referred to as ICG) (manufactured by Pharmaceutical and Medical Device Regulatory Science Society of Japan), 2 mg of iron(III) chloride hexahydrate (hereinafter, abbreviated to iron(III) chloride) (manufactured by Wako Pure Chemical Industries, Ltd.), and 80 mg of PLGA 5020 (hereinafter, abbreviated to PLGA) (manufactured by Wako Pure Chemical Industries, Ltd.) in 2 mL of DMSO (manufactured by Kishida Chemical Co., Ltd.). Herein, the amount of iron atoms was adjusted to be 1 equivalent with respect to ICG. The resulting solution was transparent and green.

### Preparation of Dispersion-Stabilizer Dispersion

One mole of 2-[4-(2-hydroxyethyl)-1-piperazinyl]ethanesulfonic acid (hereinafter, abbreviated as HEPES) (manufactured by Invitrogen) was diluted with ultrapure water to 10 mM, and NaCl was added thereto up to 0.3 M. To the resulting solution, 1.5 g of a surfactant, Pluronic F68 (manufactured by Sigma), was added to prepare a dispersion-stabilizer dispersion.

### Preparation of Particle Dispersion

The organic solvent solution was dropwise added to the dispersion-stabilizer dispersion with stirring using a magnetic stirrer to prepare a particle dispersion.

### Purification of Particle Dispersion

The resulting particle dispersion was centrifuged with a high speed microcentrifuge (MX-300, manufactured by Tomy Seiko Co., Ltd.) at 20000 g for 20 minutes at 4° C., and the supernatant was removed to remove excess supernatant. To the remaining precipitate, 10 mM HEPES was added to prepare a particle re-dispersion. This particle re-dispersion was sequentially filtered through filters with pore sizes of 5.0, 1.2, 0.8, 0.45, and 0.22 μm to prepare Particle A1.

### Example A2

Particle A2 was prepared as in Example A1 except that Pluronic F68 was replaced by polyvinyl alcohol (PVA, trade name: Mowiol 4-88, manufactured by Aldrich, rate of hydrolysis: 86.7 to 88.7% by mol) having a concentration of 4% by weight in a 10 mM HEPES solution.

### Example A3

Particle A3 was prepared as in Example A1 except that Pluronic F68 was replaced by 60 mg of dextran (molecular weight: 40 K, manufactured by Tokyo Chemical Industry Co., Ltd.).

### Example A4

Particle A4 was prepared as in Example A1 except that Pluronic F68 was replaced by 60 mg of heparin sodium (manufactured by Tokyo Chemical Industry Co., Ltd.).

### Example A5

Particle A5 was prepared as in Example A3 except that iron(III) chloride was replaced by calcium chloride (manufactured by Nacalai Tesque, Inc.).

### Example A6

Particle A6 was prepared as in Example A3 except that iron(III) chloride was replaced by magnesium chloride hexahydrate (manufactured by Kishida Chemical Co., Ltd.).

### Example A7

Particle A7 was prepared as in Example A3 except that iron(III) chloride was replaced by manganese(II) chloride tetrahydrate (manufactured by Kishida Chemical Co., Ltd.).

### Example A8

Particle A8 was prepared as in Example A3 except that iron(III) chloride was replaced by tin(II) chloride dihydrate (manufactured by Kishida Chemical Co., Ltd.).

### Example A9

Particle A9 was prepared as in Example A3 except that iron(III) chloride was replaced by zinc) chloride (manufactured by Kishida Chemical Co., Ltd.).

### Comparative Example A1

Particles were prepared as in Example A1 except that iron(III) chloride was not used.

### Comparative Example A2

Particles were prepared as in Example A1 except that PLGA was not used.

### Comparative Example A3

Particles were prepared as in Example A2 except that PLGA was not used.

Table A1 shows particle diameter and ICG utilization efficiency of each particles prepared above. The ICG utilization efficiency was calculated by dividing the ICG amount contained in the collected particles by the charged ICG amount.

TABLE A1

| | | | | | ICG utilization |
|---|---|---|---|---|---|
| | Polyvalent | Hydrophobic | | Particle | efficiency |
| | metal ion | polymer | Dispersion stabilizer | diameter [nm] | [%] |
| Particle A1 | Fe3+ | PLGA | Pluronic F68 | 260 | 64 |
| Particle A2 | | | PVA | 220 | 59 |
| Particle A3 | | | Dextran | 200 | 96 |
| Particle A4 | | | Heparin | 350 | 98 |
| Particle A5 | Ca2+ | PLGA | Dextran | 186 | 25 |
| Particle A6 | Mg2+ | PLGA | Dextran | 205 | 14 |
| Particle A7 | Mn2+ | PLGA | Dextran | 188 | 18 |
| Particle A8 | Sn2+ | PLGA | Dextran | 179 | 17 |
| Particle A9 | Zn2+ | PLGA | Dextran | 150 | 53 |

The results shown in Table A1 reveal that every particles contain a high amount of ICG.

Table A2 shows the molar absorption coefficient per particle and photoacoustic signal intensity per particle at a wavelength of 710 nm in Particles A1 to A9, and stability of Particles A1 to A9 in 90% serum.

The molar absorption coefficient per particle and the photoacoustic signal intensity per particle were calculated assuming that the particle diameter was 100 nm. Note that E+x in the Tables represents 10 to the power of X. For example, 3.6E+09 means 3.6×10⁹.

TABLE A2

| | | | | Maximum | Molar absorption | Photoacoustic |
|---|---|---|---|---|---|---|
| | | | | absorption | coefficient | signal intensity |
| | Polyvalent | Hydrophobic | Dispersion | wavelength | per particle | per particle |
| | metal ion | polymer | stabilizer | [nm] | [M*cm] | [V/J/M] |
| Particle A1 | Fe3+ | PLGA | Pluronic F68 | 790 | 3.6E+09 | 1.4E+11 |
| Particle A2 | | | PVA | 890 | 6.7E+08 | 7.1E+10 |
| Particle A3 | | | Dextran | 880 | 7.2E+08 | 1.1E+11 |
| Particle A4 | | | Heparin | 890 | 3.1E+09 | 1.7E+11 |
| Particle A5 | Ca2+ | PLGA | Dextran | 780 | 1.1E+10 | 1.3E+11 |
| Particle A6 | Mg2+ | PLGA | Dextran | 780 | 4.8E+09 | 7.6E+10 |
| Particle A7 | Mn2+ | PLGA | Dextran | 780 | 6.3E+09 | 1.1E+11 |
| Particle A8 | Sn2+ | PLGA | Dextran | 780 | 3.5E+09 | 6.2E+10 |
| Particle A9 | Zn2+ | PLGA | Dextran | 780 | 4.0E+09 | 1.3E+10 |

The results shown in Table A2 reveal that every particles highly absorb light in the near-infrared wavelength region to produce a strong photoacoustic signal.

Particles A1 to A4 and A9 and the particles produced in Comparative Example A1 were evaluated for remaining rate of coloring agent in particles in 90% serum. Table A3 shows the results. The results of the particles prepared in Comparative Examples A2 and A3 are also shown in Table A3.

TABLE A3

| | Polyvalent | | | Coloring agent |
|---|---|---|---|---|
| | metal | Hydrophobic | Dispersion | retention rate in |
| | ion | polymer | stabilizer | 90% serum |
| Particle A1 | Fe3+ | PLGA | Pluronic F68 | 23% |
| Particle A2 | | | PVA | 40% |
| Particle A3 | | | Dextran | 53% |
| Particle A4 | | | Heparin | 45% |
| Particle A9 | Zn2+ | PLGA | Dextran | 39% |
| Comparative | — | PLGA | Pluronic F68 | 16% |
| Example 1 | | | | |
| Comparative | Fe3+ | — | Pluronic F68 | No formation |
| Example 2 | | | | of particles |
| Comparative | | — | PVA | No formation |
| Example 3 | | | | of particles |

As obvious from the results of Examples A1 to A4 and A9, addition of polyvalent metal ions allows the particles to maintain a high concentration of ICG even after being left in 90% serum at 37° C. for 24 hours.

As obvious from the results of the contrast agent produced in Comparative Example A1, in particles not containing polyvalent metal ions, the remaining rate of ICG in the particles is decreased after leaving in 90% serum at 37° C. for 24 hours.

As obvious from the results of Comparative Examples A2 and A3, in the above-described process of producing particles, particles are not formed under conditions of not containing the hydrophobic polymer, even if polyvalent metal ions are contained.

As the results shown above, the particles composed of a matrix of coloring agent having an anionic functional group/polyvalent metal ion/hydrophobic polymer according to aspects of the present invention can highly retain the coloring agent having an anionic functional group, i.e., ICG, in the particles. Thus, aspects of the present invention can provide a photoacoustic imaging contrast agent that does not reduce the coloring agent content in the particles even after being left in 90% serum at 37° C. for 24 hours and can emits stronger photoacoustic signals compared with those in known technology, without reducing the coloring agent content in the particles even after being left in 90% serum at 37° C. for 24 hours.

### Example B1

ICG (3.5 mg, manufactured by Pharmaceutical and Medical Device Regulatory Science Society of Japan) was dissolved in 2.0 mL of ultrapure water. Iron(III) chloride hexahydrate (0.6 mg), which was 0.5 equivalents with respect to ICG, was dissolved in 4.0 mL of ultrapure water.

The iron chloride solution was added to the ICG solution with stirring to prepare a particle dispersion. The resulting particle dispersion was centrifuged at 20000 g for 45 minutes at 4° C. to collect particles. The collected particles were washed with ultrapure water and were then collected by centrifugation at 20000 g for 45 minutes at 4° C. The collected particles were re-dispersed in ultrapure water. The resulting particle dispersion was filtered through a filter with a pore size of 0.45 μm to prepare photoacoustic imaging contrast agent B1.

### Example B2

Photoacoustic imaging contrast agent B2 was prepared as in Example B1 except that the iron(III) chloride hexahydrate was replaced by zinc(II) chloride (manufactured by Kishida Chemical Co., Ltd.).

### Example B3

ICG (7.0 mg) was dissolved in 4.0 mL of a 10 mM 2-[4-(2-hydroxyethyl)-1-piperazinyl]ethanesulfonic acid (HEPES, manufactured by Invitrogen) buffer solution (hereinafter, may be simply abbreviated as HEPES). Iron(III) chloride hexahydrate (4.9 mg), which was 2.0 equivalents with respect to ICG, was dissolved in 8.0 mL of 10 mM HEPES. Tannic acid (13.0 mg, manufactured by Kishida Chemical Co., Ltd.), which was 1.0 equivalents with respect to ICG, was dissolved in 4.0 mL of 10 mM HEPES. The ICG solution was added to the tannic acid solution with stirring. Subsequently, the iron chloride solution was added thereto to prepare a particle dispersion. The resulting particle dispersion was centrifuged at 20000 g for 45 minutes at 4° C. to collect particles. The collected particles were washed with 10 mM HEPES and were then collected by centrifugation at 20000 g for 45 minutes at 4° C. The collected particles were re-dispersed in 10 mM HEPES. The resulting particle dispersion was filtered through a filter with a pore size of 0.45 μm to prepare photoacoustic imaging contrast agent B3.

### Example B4

Photoacoustic imaging contrast agent B4 was prepared as in Example B3 except that the iron(III) chloride hexahydrate was replaced by zinc(II) chloride.

### Example B5

ICG (3.5 mg) was dissolved in 2.0 mL of ultrapure water. Iron(III) chloride hexahydrate (0.6 mg), which was 0.5 equivalents with respect to ICG, was dissolved in 4.0 mL of ultrapure water. Dextran (6.4 mg, manufactured by Tokyo Chemical Industry Co., Ltd.), which was 0.1 equivalents with respect to ICG, was dissolved in 2.0 mL of ultrapure water. The ICG solution was added to the dextran solution with stirring. Subsequently, the iron chloride solution was added thereto to prepare a particle dispersion. The resulting particle dispersion was centrifuged at 20000 g for 45 minutes at 4° C. to collect particles. The collected particles were washed with ultrapure water and were then collected by centrifugation at 20000 g for 45 minutes at 4° C. The collected particles were re-dispersed in ultrapure water. The resulting particle dispersion was filtered through a filter with a pore size of 0.45 μm to prepare photoacoustic imaging contrast agent B5.

### Example B6

Photoacoustic imaging contrast agent B6 was prepared as in Example B5 except that the iron(III) chloride hexahydrate was replaced by zinc(II) chloride.

### Comparative Example B1

An aqueous solution of 1 mM ICG was prepared and used in experiments.

### Comparative Example B2

To ICG, human serum albumin (manufactured by Sigma-Aldrich) was added to at an amount of 2.4 equivalents with respect to ICG, and the concentration of ICG was adjusted to 1 mM to prepare complex (ICG-albumin complex).

### Evaluation

The accumulation ratios, to SLN, of the photoacoustic imaging contrast agents prepared in Examples B1 to B6 and reagents prepared in Comparative Examples B1 and B2 were calculated according to the above-described evaluation examples for accumulation in SLN. The results are shown in Table B1. In Table B1, the accumulation ratio means an accumulation rate of each photoacoustic imaging contrast agent or the ICG-albumin complex when the accumulation rate of the ICG aqueous solution used in Comparative Example B1 is assumed as 1. The wavelengths used for measuring absorbance for calculating the accumulation rates are also shown in Table B1.

Table B1 also shows the particle diameter, molar absorption coefficient (M⁻¹·cm⁻¹) per particle, and photoacoustic signal strength (V·J⁻¹·M⁻¹) per particle in the resulting photoacoustic imaging contrast agents and reagents. Note that E+x in the Table B1 represents 10 to the power of X. For example, 2.4E+10 means 2.4×10¹⁰.

TABLE B1

| | | | | | | | Wavelength | Molar | Photoacoustic |
|---|---|---|---|---|---|---|---|---|---|
| | | Coloring agent | | | | | used for | absorption | signal |
| | | having an | | | Particle | Accumu- | measuring the | coefficient | intensity |
| | | anionic | Polyvalent | Polyol | diameter | lation | absorbance | per particle | per particle |
| | | functional group | metal ion | compound | (nm) | ratio | (nm) | (M−1 cm−1) | (V J−1 M−1) |
| Example 1 | Photoacoustic | ICG | Iron(III) | Not used | 290 | 6 | 790 | 2.4E+10 | 6.5E+10 |
| | imaging contrast | | ion | | | | | | |
| | agent 1 | | | | | | | | |
| Example 2 | Photoacoustic | ICG | Zinc(II) | Not used | 397 | 14 | 800 | 3.7E+10 | 8.9E+10 |
| | imaging contrast | | ion | | | | | | |
| | agent 2 | | | | | | | | |
| Example 3 | Photoacoustic | ICG | Iron(III) | Tannic acid | 146 | 18 | 800 | 7.9E+10 | 3.1E+10 |
| | imaging contrast | | ion | | | | | | |
| | agent 3 | | | | | | | | |
| Example 4 | Photoacoustic | ICG | Zinc(II) | Tannic acid | 346 | 15 | 800 | 2.8E+10 | 4.1E+10 |
| | imaging contrast | | ion | | | | | | |
| | agent 4 | | | | | | | | |
| Example 5 | Photoacoustic | ICG | Iron(III) | Dextran | 55 | 17 | 800 | 5.4E+10 | 9.9E+10 |
| | imaging contrast | | ion | | | | | | |
| | agent 5 | | | | | | | | |
| Example 6 | Photoacoustic | ICG | Zinc(II) | Dextran | 177 | 21 | 800 | 3.9E+10 | 9.2E+10 |
| | imaging contrast | | ion | | | | | | |
| | agent 6 | | | | | | | | |
| Comparative | ICG aqueous | ICG | Not used | Not used | — | 1 | 800 | — | — |
| Example 1 | solution | | | | | | | | |
| Comparative | ICG-albumin | ICG | Not used | Not used | — | 0.7 | 800 | — | — |
| Example 2 | complex | | | | | | | | |

It was recognized from Examples and Comparative Examples that accumulation, in SLN, of a photoacoustic imaging contrast agent composed of ICG, as a coloring agent having an anionic functional group, and ions of a polyvalent metal is higher than that of ICG alone.

### Example C1

Particle C1 was prepared as in Example A1 except that PLGA 5020 and Pluronic F68 were replaced by PLGA 5005 (manufactured by Wako Pure Chemical Industries, Ltd.) and Tween 20, respectively.

### Example C2

Particle C2 was prepared as in Example A1 except that PLGA 5020 and Pluronic F68 were replaced by PLA 0020 (manufactured by Wako Pure Chemical Industries, Ltd.) and Tween 20, respectively.

### Example C3

Particle C3 was prepared as in Example A1 except that PLGA 5020 and Pluronic F68 were replaced by PLA 0005 (manufactured by Wako Pure Chemical Industries, Ltd.) and Tween 20, respectively.

Table C1 shows the polyvalent metal ion, maximum absorption wavelength, particle diameter, molar absorption coefficient per particle, remaining rate of coloring agent in particles, and IR measurement results in Particles C1 to C3. The molar absorption coefficient per particle was calculated assuming that the particle diameter was 100 nm.

### Example D1

Table D1 shows the IR measurement results of the resulting Particles 1, 2, 3, 7, 8, 9, B1, and B2.

TABLE C1

| | | | IR measure- | IR measure- |
|---|---|---|---|---|
| | | | ment results | ment results |
| | | | Characteristic | Characteristic |
| | Metal chloride | Matrix | peak 1 (cm⁻¹) | peak 2 (cm⁻¹) |
| Particle 1 | Iron(III) chloride | Tannic acid | 1032 | |
| Particle 2 | Zinc(II) chloride | Tannic acid | 1042 | — |
| Particle 3 | Aluminum(III) | Tannic acid | 1042 | — |
| | chloride | | | |
| Particle 7 | Iron(III) chloride | Dextran | 1042 | — |
| Particle 8 | Zinc(II) chloride | Dextran | 1041 | — |
| Particle 9 | Aluminum(III) | Dextran | 1032 | — |
| | chloride | | | |
| Particle B1 | Iron(III) chloride | — | — | 1165 |
| Particle B2 | Zinc(II) chloride | — | — | 1166 |
| ICG | — | — | 1038 | 1136 |

The IR measurement results show a shift in the peak derived from the coloring agent having an anionic functional group, which suggests interaction of ionic bonds, coordinate bonds, and other factors.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-227507 filed Oct. 15, 2011, No. 2011-227508 filed Oct. 15, 2011, No. 2011-227509 filed Oct. 15, 2011, and No. 2012-063899 filed Mar. 21, 2012, which are hereby incorporated by reference herein in their entirety.

What is claimed is:

1. Particles comprising a coloring agent having an anionic functional group, ions of a polyvalent metal, and a polyol compound.

2. Particles comprising a coloring agent having an anionic functional group, ions of a polyvalent metal, and a hydrophobic polymer.

3. The particles according to claim 1, wherein
the coloring agent having an anionic functional group is any one of indocyanine green, indigo carmine, and Patent Blue.

4. The particles according to claim 1, wherein
the ions of a polyvalent metal include at least any of calcium ions, magnesium ions, manganese ions, tin ions, zinc ions, and iron ions.

5. The particles according to claim 1, wherein
the polyol compound is any one of tannic acid, dextran, heparin, and heparin sodium.

6. The particles according to claim 1, the particles further comprising a dispersion stabilizer.

7. A photoacoustic imaging contrast agent comprising particles according to claim 1 and a dispersion medium.

8. A contrast agent for sentinel lymph node comprising particles according to claim 1 and a dispersion medium.

9. A photoacoustic imaging contrast agent comprising particles comprising a coloring agent having an anionic functional group and ions of a polyvalent metal.

10. A method of producing particles, the method comprising:
preparing a mixture A of a coloring agent having an anionic functional group, a polyvalent metal ion salt, a hydrophobic polymer, and an organic solvent; and
mixing the mixture A and water,
wherein the organic solvent is dimethyl sulfoxide.
